(19) United States
(12) Patent Application Publication
(10) Pub. No.: US 2021/0137837 A1
(43) Pub. Date: May 13, 2021

(54) TREATMENTS USING OXYGEN MICROBUBBLES AND CANNABIDIOL

(71) Applicant: RESPIROGEN, INC., Boulder, CO (US)

(72) Inventors: Paul A MOUNTFORD, Boulder, CO (US); Robert M SCRIBNER, Longmont, CO (US); Robert T SCRIBNER, Boulder, CO (US); Mark A. BORDEN, Boulder, CO (US)

(21) Appl. No.: 17/093,444

(22) Filed: Nov. 9, 2020

Related U.S. Application Data
(60) Provisional application No. 62/932,782, filed on Nov. 8, 2019.

(51) Int. Cl. A61K 9/107; A61K 9/00; A61K 31/05; A61K 33/00

(52) U.S. Cl. CPC A61K 9/1075; A61K 33/00; A61K 31/05; A61K 9/0014

(57) ABSTRACT

The invention relates to an improved oxygen microbubble (OMB)-and-Cannabidiol (CBD) formulation that can be administered for medical and/or therapeutic purposes in any entry route and delivery method to improve bioavailability and half-life within the body. The dual CBD-OMB emulsion/formulation described herein may be a product capable of simultaneously delivering oxygen and CBD to a target tissue of interest to also further enhance blood flow to the target tissue site potentially increasing the uptake of CBD by the anatomy and thereby enhancing CBD's bioavailability in humans.

### CROSS-REFERENCE TO RELATED APPLICATIONS

This application claims the benefit of U.S. Provisional Application No. 62/932,782 entitled “Stabilized Dual Cannabidiol Oxygen Emulsion for Oral and Topical Deliver,” filed Nov. 8, 2019, the disclosure of which is incorporated by reference herein in its entirety.

### TECHNICAL FIELD

The invention relates to methods, devices, and systems for improved microbubbles (OMB) enhanced with Cannabidiol (CBD). More specifically, the invention relates methods, devices, and systems of using OMB's enhanced with CBD to treat a patient's body and its respective tissues for variety of medical or therapeutic treatments.

### BACKGROUND OF THE INVENTION

Cannabidiol (CBD) offers potential uses for medical and therapeutic treatments, but its bioavailability within a person's body may lessen CBD delivery and/or effectiveness. The bioavailability of CBD's may depend on many factors, including but not limited to route of entry, delivery method, and/or a process known as pharmacokinetics (i.e., how compounds are processed by the body).

Currently, a variety of delivery methods exist for CBD, such as oral, inhalation, mucosal, transdermal and intravenous routes. However, these delivery methods may be less than optimal in that oil-based CBD formulations (i.e., fat-soluble) and water-soluble CBD formulations (i.e., water-and-CBD oil) may make absorption within the body a challenge, especially where maintaining consistent half-life levels within a target region and/or tissue can be difficult.

### BRIEF SUMMARY OF THE INVENTION

Various aspects of the present invention include the realization of a need for improved diagnosis and/or treatment of tissues of the human body and/or other animals, including the use of OMB's enhanced with CBD to external skin surfaces and/or internal organs of the body such as the inner ears, lungs, nasal cavities, mouth, digestive tract and/or excretory tract of a patient via pre-existing anatomical access pathways, as well as treating other tissues via natural and/or surgically created access pathways. In various embodiments, OMB's enhanced with CBD can be a particularly effective combination treating internal and/or external tissue surfaces as well as other wounds, including tissue damage resulting from and/or experiencing delayed healing due to ischemic conditions. In various embodiments, wounds and tissue injuries, including skin ulcers and/or other types of damaged skin or other tissue surfaces and/or subsurface structures, can be treated by application of a compound which includes oxygenated microbubble formulations enhanced with CBD, which may be applied using a variety of delivery modalities and/or treatment algorithms to desirably promote shorter time to healing.

### BRIEF DESCRIPTION OF THE SEVERAL VIEWS OF THE DRAWINGS

The accompanying drawings, which are included to provide a further understanding of the invention and are incorporated in and constitute a part of this specification, illustrate embodiments of the invention and together with the description serve to explain the principles of the invention:

FIG. 1 depicts one exemplary embodiment of a CBD molecule being embedded within the phospholipid monolayer shell of an OMB;

FIG. 2 depicts an embodiment of functionalized CBD molecules attached to functionalized pegylated lipid molecules;

FIG. 3 depicts another embodiment of OMBs suspended in a continuous CBD oil and water medium;

FIG. 4 depicts a schematic of how an alternative embodiment of a dual CBD nanodroplet-oxygen microbubble emulsion can be suspended in a water continuous phase; and

FIG. 5 depicts an embodiment of functionalized CBD nanodroplets conjugated to the functionalize surface of an oxygen microbubble.

### DETAILED DESCRIPTION OF THE INVENTION

Cannabidiol (CBD) is one of the active ingredients in cannabis which is derived from the hemp plant. In oral and transdermal animal models, CBD has shown the potential of being used as an anti-inflammatory. Specifically, CBD may be capable of aiding patients with colitis, collagen-induced arthritis, neuroinflammation, neutrophil chemotaxis, ischemia-reperfusion injury and acute lung injury. CBD can be purchased in oil, solid, pill, and beverage form for oral and/or topical administration. Recently, CBD has been formulated as a shell-stabilized oil-in-water nanoemulsion for increased bioavailability via oral ingestion. The oil-in-water CBD nanoemulsion, commonly referred to a water-soluble CBD, showed an estimated bioavailability of 93.9% in rats whereas plain CBD oil only had a 73.3% bioavailability in rats. It is estimated that CBD oil only has a bioavailability of 6% in humans.

Shell-stabilized oxygen microbubbles (OMB) have been utilized for delivering oxygen within the anatomy in animals independent of the lungs. OMB can provide oxygen to local tissues upon administration. An increase in oxygen presence may improve local blood flow to the respective tissues. The technology presented herein describes a dual CBD-OMB oil/gas-in-water emulsion for the purpose of delivering both CBD and oxygen to tissue. Specifically, the described dual emulsion herein will provide CBD to the anatomy while simultaneously co-delivering oxygen for improving blood flow in the tissue at the site of delivery.

Although CBD has the potential to treat various forms of inflammation, its low bioavailability in humans inhibits its ability to treat inflammation at reasonable oral or transdermal dose. Currently, the bioavailability of CBD in humans is approximately 6%. Delivering CBD in the “water-soluble” nanoemulsion form may increase its bioavailability to approximately 9-10%. The dual CBD-OMB emulsion looks to further increase the bioavailability of CBD in the human anatomy by introducing oxygen simultaneously with CBD to the tissue. The presence of oxygen at the site of delivery may be able to increase blood flow and improve the uptake of CBD. As an example, but not limited to, the technology described herein could be administered orally in the form of a liquid beverage. This liquid beverage could consist of the dual emulsion described in FIG. 4. Once the dual CBD-OMB emulsion hits the digestive tissue, the blood flow to the digestive track may increase and the CBD nanodrops may experience uptake larger than 9-10%.

As a result, the need exists to create an oxygen microbubbles (OMB)-and-Cannabidiol (CBD) formulation that can be administered for medical and/or therapeutic purposes in any entry route and delivery method to improve bioavailability and half-life within the body. The dual CBD-OMB emulsion/formulation described herein may be a product capable of simultaneously delivering oxygen and CBD to a target tissue of interest for the purpose of providing a therapeutic (anti-inflammatory as an example but not limited to) to/within the human anatomy. Additionally, this product's oxygen may enhance blood flow to the target tissue site potentially increasing the uptake of CBD by the anatomy and thereby enhancing CBD's bioavailability in humans.

The technology described herein depicts many different ways in which CBD and OMB can be co-administered as a single stabilized dual emulsion. The first method for formulating and utilizing a dual CBD-OMB emulsion is depicted in FIG. 1. FIG. 1 shows the CBD molecule being embedded within the phospholipid monolayer shell of the OMB. Here, the stabilizing phospholipid monolayer shell of the OMB is used to embed the CBD. Specifically, the CBD and phospholipid shell materials are prepared together (at a molar ratio of 2:8 as an example but not limited to) prior to microbubble formulation. Upon microbubble formulation, the CBD molecules in combination with the phospholipid molecules structure themselves around the oxygen gas creating a micron-sized oxygen gas bubble stabilized in water by a phospholipid-CBD shell.

FIG. 2 illustrates how functionalized CBD molecules are attached to functionalized pegylated lipid molecules. The second technology described herein utilizes functionalized CBD and pegylated phospholipid molecules for the active binding of CBD to the microbubble shell. As an example, but not limited to, oxygen microbubbles can be formed with a phospholipid shell containing approx. 10-20% (by molar volume) biotin functionalized pegylated phospholipids. Upon microbubble formation, CBD molecules functionalized with avidin groups are mixed with the biotinylated OMBs. The end result is an OMB that has CBD conjugated to the OMB surface via avidin-biotin binding.

FIG. 3 depicts a diagram that shows OMBs suspended in a continuous CBD oil and water medium. The third and most simple way of producing a dual CBD-OMB emulsion is to mix phospholipid-stabilized OMBs with CBD oil dispersed in water.

FIG. 4 depicts a schematic of how a dual CBD nanodroplet-oxygen microbubble emulsion where both emulsions are suspended in a water continuous phase. This is a fourth method for delivering CBD and oxygen to the anatomy simultaneously is to mix a “water-soluble” CBD nanoemulsion with the OMB emulsion so that both emulsions exist together suspended in a continuous water phase. This fabrication of the dual CBD-OMB emulsion is depicted in FIG. 4.

FIG. 5 depicts a schematic showing functionalized CBD nanodroplets conjugated to the functionalize surface of the oxygen microbubble. The final method for co-administering CBD and oxygen is depicted in FIG. 5 where shell-stabilized functionalized CBD nanodroplets have been conjugated (attached) to the surface of a functionalized oxygen microbubble shell.

In various embodiments, the CBD nanodroplet-oxygen microbubble emulsions and/or other microbubble products described herein can be applied to a patient via various routes, including oral, inhalation, mucosal, transdermal and/or intravenous routes, including treatments to external skin surfaces and/or internal organs of the body such as the inner ears, lungs, nasal cavities, mouth, digestive tract and/or excretory tract of a patient via pre-existing anatomical access pathways, as well as treating other tissues via natural and/or surgically created access pathways.

Various embodiments described herein could also have particular utility with regards to various types of damaged and/or injured surface and/or subsurface skin tissues, including surface/subsurface skin tissue burns due to excessive heat, excessive cold, chemical contact, radiation effects, wind abrasion and/or otherwise induced tissue damage, including bacterial damage. It should be understood that the various assessment and/or treatment modalities described herein could be utilized in conjunction with the treatment and/or management of such wounds, including various combinations of the various embodiments disclosed herein.

In various alternative embodiments, the various microbubble formulations described herein may incorporate alternative gases to oxygen, such as carbon dioxide, nitrogen, and/or other gases.

If desired, the various OMB formulation described herein can facilitate various levels of oxygen and/or carbon dioxide exchange across various the tissues in and/or proximate to a targeted wound bed (potentially including across cellular boundaries of a wide variety of body tissues). Moreover, the introduction of CBD-OMBs to a surface and/or subsurface of a wound bed is not subject to a strict upper limit of the microbubble size and volume fraction, since the microbubbles are not intended to be directly injected into a vascular channel (in many cases) and thus can safely ripen, burst or otherwise degrade and/or be removed from a target anatomy as desired. These methods desirably provide oxygen supply and/or carbon dioxide removal to a subject to facilitate healing of the wound and/or other tissues.

### Other Joints, Organs and Tissues

The various embodiments described herein, including the treatments thereof using various tools, techniques and surgical methods can be applied to various tissues in a human or animal body, including any soft or hard tissues including, without limitation, joint tissues, a spine, an elbow, a shoulder, a wrist, a hand, a finger, a jaw, a hip, a knee, an ankle, a foot, or a toe joint. In a similar manner, various alternative embodiments and/or modifications thereof could be used for the treatment of soft tissue structures and/or other organs, including organs/structures within the body such as the lungs, heart, heart tissue grafts and/or heart transplants, and/or structures within the body and/or digestive tracts such as internal stomach surfaces (i.e., stomach ulcers), intestinal lesions, hemorrhoids, and/or ulcers of the small and large intestines, etc.

### Headings

The headings provided herein are merely for the reader's convenience and should not be construed as limiting the scope of the various disclosures or sections thereunder, nor should they preclude the application of such disclosures to various other embodiments or sections described herein.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention. The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the claims provided herein

What is claimed is:

1. A method of improving the transfer of cannabidiol across a cellular membrane comprising;
contacting an outer surface of the cellular membrane with an aqueous formulation comprising cannabidiol and microbubbles containing oxygen.

2. The method of claim 1, wherein the microbubbles are formulated from a lipid.

3. The method of claim 1, wherein the microbubbles are formulated from a polymer.

4. The method of claim 1, further comprising the step of applying the aqueous formulation to a surface of an external skin layer.

5. The method of claim 1, further comprising the step of applying the aqueous formulation to a surface layer of lung tissue.
